(19) United States
(12) Patent Application Publication
(10) Pub. No.: US 2007/0055323 A1
(43) Pub. Date: Mar. 8, 2007

(54) Calibration of Cochlear Implant Dynamic Range

(75) Inventors: Donald K. Eddington, Belmont, MA (US); Barbara Herrmann, Walpole, MA (US)

Correspondence Address: FISH & RICHARDSON PC, P.O. BOX 1022, MINNEAPOLIS, MN, 55440-1022, US

(73) Assignee: MASSACHUSETTS EYE & EAR INFIRMARY, Boston, MA (US)

(21) Appl. No.: 11/470,449

(22) Filed: Sep. 6, 2006

Related U.S. Application Data
(60) Provisional application No. 60/714,504, filed on Sep. 6, 2005.

(51) Int. Cl. A61N 1/00

(52) U.S. Cl. 607/59

(57) ABSTRACT

A computer-readable medium has, encoded thereon, software for executing a method for detecting coupling between electrodes of a neural prosthesis implanted to stimulate neural tissue. The software includes instructions for stimulating a first electrode of the prosthesis with a first test signal having a first frequency; stimulating a second electrode of the prosthesis with a second test signal at a second frequency that differs from the first frequency; and identifying an occurrence of a beat signal indicative of an interaction between the first and second test signal.

### RELATED APPLICATIONS

Under 35 USC 119, this application claims the benefit of the priority date of U.S. Provisional Application 60/714,504, filed on Sep. 6, 2005, the contents of which are herein incorporated by reference.

### STATEMENT OF FEDERALLY SPONSORED RESEARCH

This invention was made with Government support under grant NIH-NIDCD N01DC 21001 awarded by the National Institute of Health. The Government may have certain rights in the invention.

### FIELD OF INVENTION

The invention is related to electrode arrays for neural stimulation, and in particular, to cochlear implants.

### BACKGROUND

A cochlear implant includes an intra-cochlear portion that follows the spiral path defined by the cochlea. Along this intra-cochlear portion lies an array of electrodes. Stimulation of an electrode excites nerve fibers along a particular segment of the spiral path.

It is desirable to stimulate an electrode with enough energy to excite nearby nerve fibers, but not so much energy that nerve fibers beyond a desired region are also stimulated. The spill-over of stimulation beyond the desired region of excitation causes distortion.

One way to reduce distortion is to reduce the amplitude of excitation at a particular electrode. However, it is difficult to determine precisely how high the amplitude of excitation can be without eliciting significant distortion.

One method commonly used to determine the maximum excitation allowed for each electrode is to simply stimulate the electrode and ask the patient how it sounds. The electrode is first stimulated weakly and the intensity increased until the patient just hears a sound. Then, the electrode is stimulated with greater intensity until a maximum comfort level is reached. These two stimulus levels define the dynamic range of stimulus level for that electrode.

A difficulty associated with the foregoing method lies in its subjectivity. The method cannot be used, for example, with children who are too young to speak. Nor does it necessarily result in an absence of distortion, since there is no guarantee that the maximum comfort level is reached prior to the onset of distortion from sources like cross-turn coupling.

### SUMMARY

The present invention provides behavioral and objective measures of whether a stimulus level introduces distortion in a signal provided by a neural sensor to the brain. This provides a basis for selecting a maximum allowable stimulus amplitude by providing a warning that a stimulus amplitude in excess of the maximum is likely to induce distortion.

In one aspect, the invention features a method for detecting coupling between electrodes of a neural prosthesis implanted to stimulate neural tissue. Such a method includes stimulating a first electrode of the prosthesis with a first test signal having a first frequency; stimulating a second electrode of the prosthesis with a second test signal at a second frequency that differs from the first frequency; and identifying an occurrence of a beat signal indicative of an interaction between the first and second test signal.

Embodiments of the invention include those in which identifying an occurrence of a beat signal includes identifying the occurrence of the beat signal on the basis of a patient's perception of the beat signal, for example, by obtaining information indicative of such perception, as well as those in which identifying an occurrence of a beat signal includes identifying the occurrence of the beat signal on the basis of an evoked potential or a stimulus artifact detected by a third electrode.

Another aspect of the invention features a method for calibrating electrodes in an electrode array. Such a method includes stimulating a test electrode at a selected stimulus level, detecting a signal indicative of coupling between discontinuous regions of neural tissue; and adjusting the selected stimulus level to avoid such coupling.

In some embodiments, detecting a signal indicative of coupling between discontinuous regions of neural tissue includes recording response signals provided by a plurality of remaining electrodes; and on the basis of the response signals, determining whether such coupling has occurred.

Other embodiments include those in which detecting coupling between discontinuous regions of neural tissue includes detecting a signal indicative of such coupling on the basis of a patient's perception of distortion, for example, by obtaining information indicative of such perception.

In another aspect, the invention features a method for identifying coupling between discontinuous regions of neural tissue. Such a method includes stimulating a first electrode of the implant with a first test signal; stimulating a second electrode of the implant with a second test signal having a second amplitude; and detecting a signal indicative of an interaction between the first and second signal.

In some embodiments, stimulating a first electrode includes selecting a characteristic of the first test signal on the basis of a patient's perception of sound.

In other embodiments, detecting a signal indicative of an interaction includes detecting the signal on the basis of a patient's perception of the interaction.

In yet other embodiments, detecting a signal indicative of an interaction includes detecting the signal on the basis of an evoked potential or a stimulus artifact detected by a third electrode.

Some embodiments also include selecting the first and second test signals to have either different frequencies, different amplitudes, or different complex amplitudes.

In another aspect, the invention includes a computer-readable medium having encoded thereon software for carrying out any of the foregoing methods.

While the invention is described herein in the context of cochlear implants, the same or similar methods, and software for carrying out those methods, can be applied to other neural prostheses.

These and other features of the invention will be apparent from the following detailed description and the accompanying figures, in which:

### BRIEF DESCRIPTION OF THE DRAWINGS

FIG. 1 is a cross-section of the cochlea showing an intra-cochlear portion of a cochlear implant passing therethrough; and

FIG. 2 is a plot of a beat threshold associated with each test electrode of the implant in FIG. 1.

FIG. 3 shows evoked potentials measured at various recording electrodes; and

FIGS. 4 and 5 show the effect of masking on coupling between electrodes.

### DETAILED DESCRIPTION

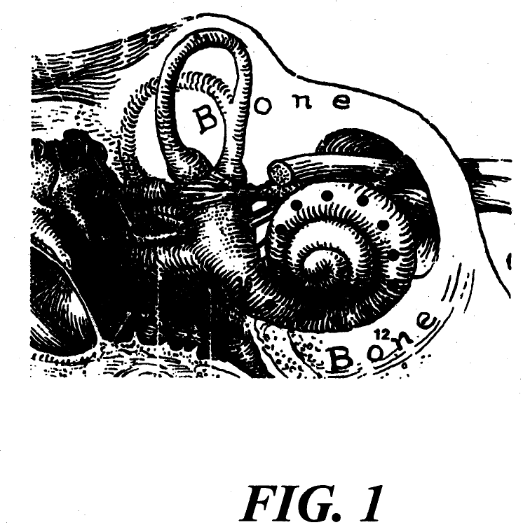

FIG. 1 shows an implant inserted into a cochlea. The particular implant has sixteen electrodes. Each electrode is adjacent to a portion of the cochlea. A region associated with the adjacent portion shall be referred to as the “excitation region” associated with that electrode.

Stimulation of an electrode results in excitation of nearby nerve fibers. Ideally, these nerve fibers are all within the excitation region of the electrode. However, in some cases, the nerve fibers excited by an electrode can extend beyond that electrode's excitation region and into excitation regions of adjacent electrodes.

Because bone is so much more resistive than either neural tissue or the fluids that fill the cochlea, current caused by an excited electrode tends to flow along the neural tissue and along the fluid-filled cochlea. As it does so, it excites nearby auditory nerve fibers along the cochlea's spiral path. As the stimulus level increases, the region of excitation expands in a regular fashion, recruiting adjacent fibers as it does so. However, a stimulus of sufficient amplitude can cause current to flow along other paths and excite regions of fibers that are not contiguous with those nearest the stimulated electrode. For instance, the current might flow across the bone and into the adjacent turn of the spiral. It would then stimulate auditory nerve fibers on the adjacent turn of the spiral.

For example, in FIG. 1, excitation of a first electrode can result in stimulation of nerve fibers adjacent to a second electrode, which is on a different turn of the spiral. This type of coupling, referred to as “cross-turn coupling,” causes distortions that may make it especially difficult for a patient to understand speech. This is because stimulation of spatially different portions of the neural tissue within the cochlea results in perception of different frequencies of sound.

Cross-turn coupling is an example of two areas of neural tissue that are not contiguous with each other being stimulated by an electrode that is intended to simulate only one of the two areas. Such stimulation will be referred to herein as the “stimulation of, or coupling between, discontiguous regions of neural tissue,” or “discontiguous stimulation.” While the methods disclosed herein has been developed in the context of cochlear implants, they are suitable for detecting the onset of such discontiguous stimulation in other applications.

A first method for determining whether a stimulus will cause such discontiguous stimulation includes applying a first periodic signal to a first electrode, and applying a second periodic signal to a second electrode. The two periodic signals have respective first and second fundamental frequencies. These two signals can thus interact to generate a signal that warbles with a beat frequency equal to the difference between the fundamental frequencies of the two signals. The detection of a warbling signal thus provides a basis for detecting discontiguous stimulation.

The warbling signal can be detected in a variety of ways. One way is to rely on the patient's perception. In this case, the amplitude of the second periodic signal is adjusted upward until the patient reports the onset of warbling, or downward until the patient reports the disappearance of warbling. A level just below that at which the patient first perceives warbling would then be a stimulus level for that electrode that minimizes distortion. This procedure is repeated for each electrode.

Other ways to detect the warbling signal include using one of the remaining electrodes in the implant as a third electrode for detecting either an evoked potential or a stimulus artifact. In either case, a characteristic of the signal from the third electrode is analyzed to identify the existence of a beat frequency. One such characteristic is the signal spectrum. Existence of a beat frequency would then indicate that the level of the applied stimuli is high enough to cause discontiguous stimulation.

The stimulus artifact is essentially what remains of the stimulus signal after it has propagated directly from its source, which would be the first and second electrodes, to the third electrode. This differs from detecting an evoked potential, which involves detecting a signal propagating on the nerve fibers themselves.

A second method of detecting the onset of discontiguous stimulation is to stimulate a test electrode and to use the remaining electrodes as sensors. These remaining electrodes thus detect the response to the stimulus in their immediate vicinity. The response can either be an evoked potential or a stimulus artifact as discussed in connection with the first method. An alternative way to practice the second method is to substitute the patient's subjective perception for the remaining electrodes.

In those cases in which the response is an evoked potential or stimulus artifact, one increases the stimulus amplitude of the test electrode while observing the response from the remaining electrodes. In a cochlear implant, when the stimulus amplitude exceeds a cross-turn coupling threshold, the observed responses will reveal the existence of cross-turn coupling between the test electrode and at least one of the remaining electrodes. On the basis of the received responses from the remaining electrodes, one determines a maximum stimulus for the test electrode.

In those cases that rely on a patient's subjective perception, the stimulus amplitude of the test electrode is increased until the patient first reports distortion. Alternatively, the stimulus amplitude can begin at a level high enough to cause distortion, in which case the amplitude is decreased until the patient reports the disappearance of distortion. In either case, the maximum stimulus is that just below that associated with the onset of distortion.

A stimulus that is below the cross-turn coupling threshold results in a plot in which the detected responses at each of the recording electrodes form a monotonically decreasing curve. In this plot, those electrodes that are furthest along on the implant from the test electrode detect a lower amplitude response than those electrodes that are closest to the test electrode. This is consistent with the idea that as the separation between the test and recording electrodes along the spiral increases, any coupling between the test electrode and the recording electrode will diminish.

A stimulus that is at or above the cross-turn coupling threshold results in an inflection point on the curve. This inflection point indicates that current has crossed over to an adjacent turn of the spiral.

Data indicative of coupling, particularly cross-turn coupling, can also take the form of a spatial distribution of intra-cochlear evoked potentials. Such data can be obtained by applying a stimulating signal to one electrode (a “stimulating electrode”) and observing responses at the remaining electrodes (“recording electrodes”) along the implanted array of electrodes. These responses are indicative of intra-cochlear evoked potentials in the vicinity of recording electrodes. The amplitudes of the intra-cochlear evoked potentials can then be plotted as a function of the recording electrode position or as a number identifying the recording electrode. In another application, a masking signal can be applied to one electrode (a “masking electrode”) concurrently with a stimulating signal at another electrode (the “stimulating electrode”). The masking signal is typically below threshold when stimulated alone and out-of-phase relative to the stimulating electrode. The concurrent application of a masking signal and a stimulating signal measures the extent to which the evoked potential elicited by stimulating one electrode as measured on a given recording electrode is influenced by adding a stimulus at a second electrode.

A third method of detecting discontiguous stimulation is to excite a first electrode with a first signal and a second electrode with a second signal that is identical to the first signal, with the exception that it has been weighted by a weighting coefficient. The weighting coefficient can be real, in which case the second signal is a scaled replica of the first signal, or it can be complex, in which case the second signal is a phase shifted and scaled replica of the first signal. The first signal is selected to have an amplitude that causes the patient to perceive a sound. Preferably, the sound is loud, but not uncomfortably so. The second signal is weighted so that its amplitude is below a threshold of neural stimulation. Once again, one would expect the measured responses to vary monotonically with distance from the source of the stimulus, with any variations from monotonicity being indicative of possible discontiguous stimulation.

Like the first and second methods, detection of discontiguous stimulation can be carried out subjectively, by asking the patient to report the incidence of distortion, or objectively, by detecting either an evoked potential or a stimulus artifact using one of the unused electrodes.

### EXAMPLE 1

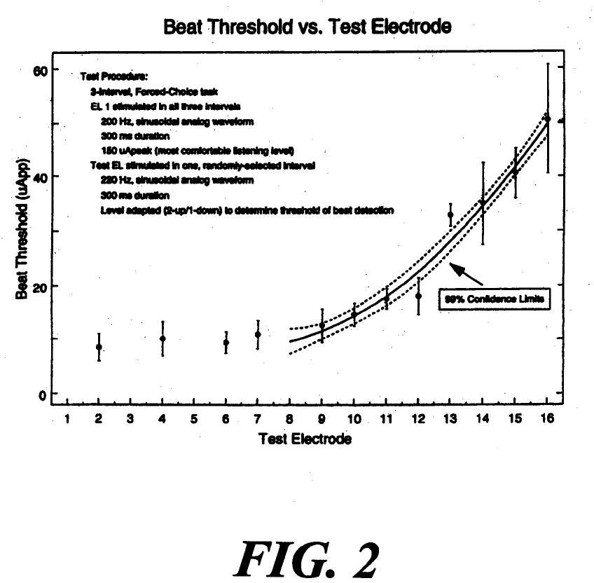

A cochlear implant with sixteen electrodes, similar to that shown in FIG. 1, was implanted in a subject. A reference electrode, which in this case was the first electrode, was stimulated by a first sinusoidal signal having a 200 Hz fundamental frequency and an amplitude of 150 microamps, which produced a steady sound sensation. A test electrode, in this case the second electrode, was stimulated by a second sinusoidal signal having a 220 Hz fundamental frequency. The amplitude of the pulses in the second signal were increased until the subject detected a warbling in the sound sensation. The amplitude at which this occurred represents a beat threshold for that electrode. This process was repeated for each of the fifteen test electrodes. The resulting fifteen beat thresholds are shown in FIG. 2.

As expected, the beat threshold increased monotonically as the distance between the test electrode and the reference electrode increased. However, a point of inflection was observed between the twelfth and thirteenth electrodes. This is believed to represent cross-turn coupling between the first electrode and the twelfth electrode.

### EXAMPLE 2

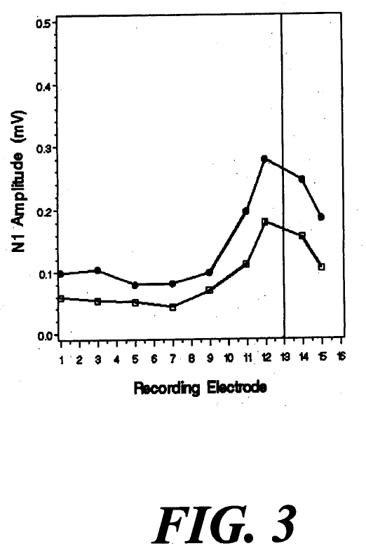

FIG. 3 shows a plot of intra-cochlear evoked potentials for two different stimuli. Filled circles represent responses to a stimulus of 400 μA; open squares represent responses to a stimulus of 480 μA. The stimulating electrode was electrode E13 (marked by a vertical line). As expected, the amplitude of the evoked potential was largest for recording electrodes closest to the stimulating electrode. In this particular subject, the decrease in evoked-potential amplitude was quite steep initially but eventually flattened and then increased at recording electrode E3 (a position consistent with cross-turn coupling) for the higher stimulus level (filled circles). Note that at the lower stimulus level (open squares), the magnitude of this inflection diminished. This type of measurement could be used to identify the maximum stimulus level at El 3 consistent with avoiding cross-turn distortion.

### EXAMPLE 3

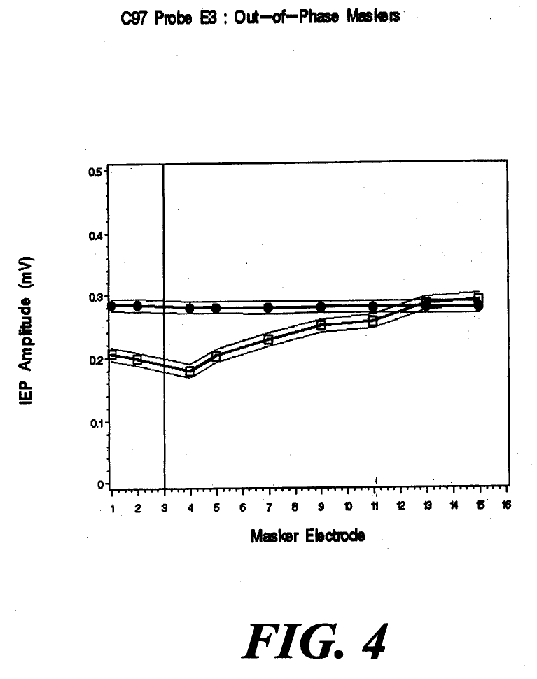

In FIG. 4, filled circles represent the intra-cochlear evoked potential magnitudes recorded on electrode E1 when electrode E3 was stimulated at a level that produced a loud (but comfortable) listening level and each of the masker electrodes were stimulated at 0 μA (i.e., the control condition). The lines above and below each curve represent the 95% confidence intervals associated with each measure.

As expected, the intra-cochlear evoked potential magnitude recorded on electrode E1 varied very little when the zero-level stimulus was applied to the various masking electrodes.

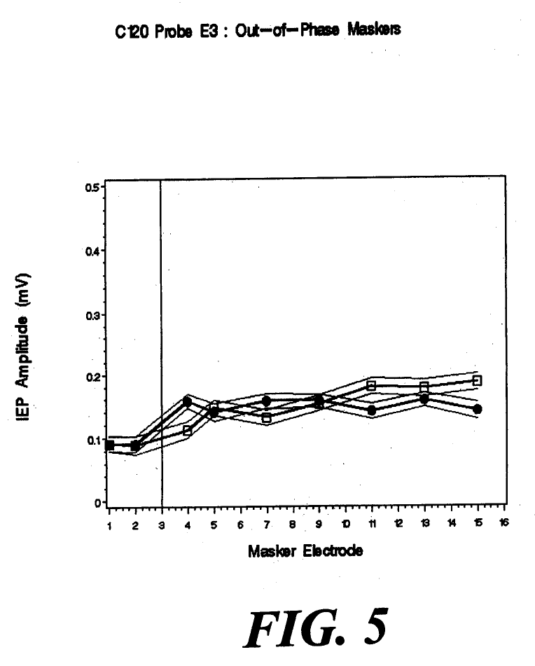

The open circles of FIG. 5 mark the intra-cochlear evoked potential magnitudes recorded on electrode E1 for the same electrode E3 stimulus, but now with an out-of-phase masking presented at the same time on one masking electrode. The amplitude of the masking signal was low enough to avoid eliciting any spike activity if delivered alone.

Note that when the masking electrode is electrode E4, the intra-cochlear evoked potential magnitude drops substantially. That is because the weak out-of-phase masking delivered at electrode E4 tended to cancel the stronger stimulus delivered to electrode E3. This resulted in an overall stimulus that was weaker than that obtained when the masking level was set to zero. The weaker overall stimulus resulted in a smaller intra-cochlear evoked potential magnitude for the “masked” condition than with the “unmasked” condition.

Note also that the impact of the masking stimulus tended to decrease as the masking electrode is moved farther from electrode E3. This is consistent with the idea that the farther away from an electrode that an out-of-phase masking stimulus is delivered, the smaller will be its impact on that electrode.

Results from a similar experiment, performed on a different patient, are summarized in FIG. 5. In this case, the electrode E3 again received the stronger stimulus and the recording electrode was again electrode E1. In this patient there was more variability in the recordings made on electrode E1 as the zero-amplitude masking was moved from electrode to electrode. Note also that the masking stimulus had a relatively small impact for masking electrodes close to electrode E3.

The most interesting impact of masking occurred when the masking signal was applied to electrodes E11, E13 and E15. In these cases, the intra-cochlear evoked potential magnitude when the out-of-phase masking signal was applied proved to be greater than it was when no masking signal was applied. This is consistent with cross-turn coupling, in which the effective phase of the masking stimulus can be reversed because of the relative geometry of the stimulating and recording electrodes.

Having described the invention, and a preferred embodiment thereof, what is claimed as new, and secured by letters patent is:

1. A computer-readable medium having encoded thereon software for identifying coupling between electrodes of a neural prosthesis, the software comprising instructions for:
stimulating a first electrode of the prosthesis with a first test signal having a first frequency;
stimulating a second electrode of the prosthesis with a second test signal at a second frequency that differs from the first frequency; and
identifying an occurrence of a beat signal indicative of an interaction between the first and second test signal.

2. The computer-readable medium of claim 1, wherein the instructions for identifying an occurrence of a beat signal comprise instructions for receiving information indicative of a patient's perception of the beat signal.

3. The computer-readable medium of claim 1, wherein the instructions for identifying an occurrence of a beat signal comprise instructions for identifying the occurrence of the beat signal on the basis of an evoked potential detected by a third electrode.

4. The computer-readable medium of claim 1, wherein the instructions for identifying an occurrence of a beat signal comprise instructions for identifying the occurrence of the beat signal on the basis of a stimulus artifact detected by a third electrode.

5. The computer-readable medium of claim 1, wherein the software further comprises instructions for receiving information from a cochlear implant.

6. A computer-readable medium having encoded thereon software for calibrating electrodes in a neural prosthesis, the software comprising instructions for:
stimulating a test electrode at a selected stimulus level;
detecting a signal indicative of coupling between discontiguous regions of neural tissue; and
adjusting the selected stimulus level to avoid the coupling between discontiguous regions of neural tissue.

7. The computer-readable medium of claim 6, wherein the instructions for detecting a signal indicative of coupling between discontiguous regions of neural tissue comprise instructions for:
recording response signals provided by a plurality of remaining electrodes; and
on the basis of the response signals, determining that coupling between discontiguous regions of neural tissue has occurred.

8. The computer-readable medium of claim 6, wherein the instructions for detecting coupling between discontiguous regions of neural tissue comprise instructions for receiving information indicative of a patient's perception of distortion, and on the basis of the information, detecting a signal indicative of coupling between discontiguous regions of neural tissue.

9. The computer-readable medium of claim 6, wherein the software further comprises instructions for receiving information indicative of a patient's perception of distortion from a cochlear implant.

10. A computer-readable medium having encoded thereon software for identifying coupling between discontiguous regions of neural tissue, the software comprising instructions for:
stimulating a first electrode with a first test signal;
stimulating a second electrode with a second test signal; and
detecting a signal indicative of an interaction between the first test signal and the second test signal.

11. The computer-readable medium of claim 10, wherein the instructions for stimulating a first electrode comprise instructions for obtaining information indicative of a patient's perception of sound, and stimulating the first electrode with a first test signal having an amplitude selected on the basis of the information.

12. The computer-readable medium of claim 11, wherein the instructions for detecting a signal indicative of an interaction comprise instructions for obtaining information indicative of a patient's perception of the interaction, and detecting the signal on the basis of the information.

13. The computer-readable medium of claim 10, wherein the instructions for detecting a signal indicative of an interaction comprise instructions for detecting the signal on the basis of an evoked potential detected by a third electrode.

14. The computer-readable medium of claim 10, wherein the instructions for detecting a signal indicative of an interaction comprise instructions for detecting the signal on the basis of a stimulus artifact detected by third electrode.

15. The computer-readable medium of claim 10, wherein the software further comprises instructions for selecting the first and second test signals to have different frequencies.

16. The computer-readable medium of claim 10, wherein the software further comprises instructions for selecting the first and second test signals to have different amplitudes.

17. The computer-readable medium of claim 10, wherein the software further comprises instructions for selecting the first and second test signals to have different complex amplitudes.

18. A method for identifying coupling between electrodes of a neural prosthesis, the method comprising:
stimulating a first electrode of the prosthesis with a first test signal having a first frequency;
stimulating a second electrode of the prosthesis with a second test signal at a second frequency that differs from the first frequency; and
identifying an occurrence of a beat signal indicative of an interaction between the first and second test signal.

19. A method for calibrating electrodes in a neural prosthesis, the method comprising:
stimulating a test electrode at a selected stimulus level;
detecting a signal indicative of coupling between discontiguous regions of neural tissue; and
adjusting the selected stimulus level to avoid coupling between discontiguous regions of neural tissue.

20. A method for identifying coupling between discontiguous regions of neural tissue, the method comprising:
stimulating a first electrode with a first test signal;
stimulating a second electrode with a second test signal; and
detecting a signal indicative of an interaction between the first and second signal.
